(19) United States
(12) Patent Application Publication
(10) Pub. No.: US 2006/0190062 A1
(43) Pub. Date: Aug. 24, 2006

(54) System and method for reducing shivering when using external cooling pads

(76) Inventor: William J. Worthen, Coto de Caza, CA (US)

Correspondence Address: ROGITZ & ASSOCIATES, 750 B STREET, SUITE 3120, SAN DIEGO, CA, 92101, US

(21) Appl. No.: 11/064,186

(22) Filed: Feb. 23, 2005

(51) Int. Cl. A61F 7/00; A61F 7/12

(52) U.S. Cl. 607/96

(57) ABSTRACT

Heat exchange pads are externally applied to a patient to induce therapeutic hypothermia. Anti-shivering agents may be administered to reduce shivering.

### I. FIELD OF THE INVENTION

The present invention relates generally to therapeutic hypothermia.

### II. BACKGROUND OF THE INVENTION

Mild or moderate hypothermia may be induced to improve the outcomes of patients suffering from such maladies as stroke, cardiac arrest, myocardial infarction, traumatic brain injury, and high intracranial pressure. The present assignee has covered one or more of the above treatments using an intravascular heat exchange catheter in U.S. Pat. Nos. 6,149,670, 6,290,717, 6,432,124, 6,454,793, 6,682,551, and 6,726,710 (collectively, “the Alsius treatment patents”), all of which are incorporated herein by reference.

As recognized herein, shivering may accompany hypothermia, which not only can lead to patient discomfort but can also counter the therapy by warming the patient. U.S. Pat. Nos. 6,702,839, 6,582,457, and 6,231,594 (collectively, “the anti-shivering patents”), incorporated herein by reference, disclose various drugs and external heating blankets for alleviating such shivering in the context of intravascular heat exchange catheters. However, as also recognized herein, these latter three patents appear to fail to envision that hypothermia may be induced by means of externally applied pads such as those disclosed in U.S. Pat. Nos. 6,827,728, 6,818,012, 6,802,855, 6,799,063, 6,764,391, 6,692,518, 6,669,715, 6,660,027, 6,648,905, 6,645,232, 6,620,187, 6,461,379, 6,375,674, 6,197,045, and 6,188,930 (collectively, “the external pad patents”), all of which are incorporated herein by reference but none of which appear to recognize that shivering might occur when using external pads, much less what to do about it. The present invention rectifies this shortfall in the prior art.

### SUMMARY OF THE INVENTION

A method for treating a patient includes disposing at least one heat exchange pad against the external skin of the patient and exchanging heat with the patient using the pad to cool the patient by lowering the body temperature of the patient. The cooling can be done on a febrile patient to maintain normothermia or it can be done to induce therapeutic hypothermia in the patient. The method also includes administering at least one anti-shivering agent to the patient by, e.g., injecting a substance into the bloodstream of the patient. The method may also include diagnosing the patient as having had cardiac arrest or myocardial infarction or stroke, prior to inducing hypothermia.

In another aspect, a system includes at least one pad that is configured for external placement on the skin of a patient and that is operable to cool the patient to induce hypothermia in the patient. A substrate is associated with the pad and bears instructions to administer an anti-shivering agent to the patient.

In still another aspect, a method includes providing instructions to a medical practitioner. The instructions include placing a heat exchange pad or pads against the skin of a patient and inducing hypothermia in the patient using the pad or pads. The method also includes countering shivering in the patient.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a schematic view of a non-limiting system in accordance with the present invention; and

FIG. 2 is a flow chart of a non-limiting method in accordance with the present invention.

### DETAILED DESCRIPTION OF THE PREFERRED EMBODIMENT

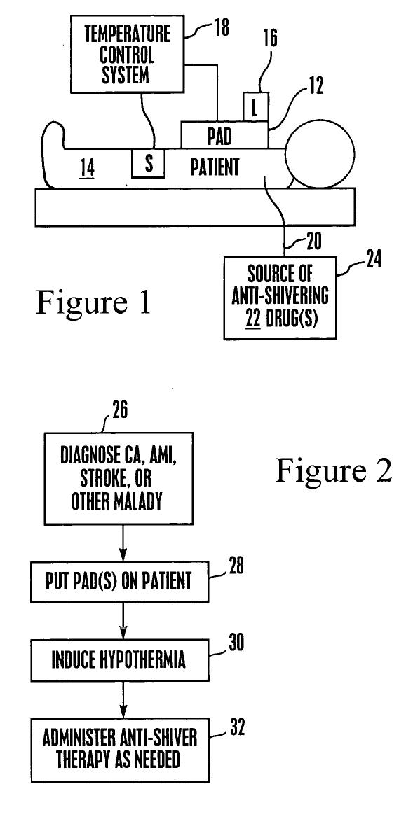

Referring initially to FIG. 1, a system is shown, generally designated 10, that includes one or more pads 12 that are positioned against the external skin of a patient 14 (only one pad 12 shown for clarity). The pad 12 is any one of the pads disclosed in the external pad patents. A substrate 16 such as a label that is affixed to the pad or an instruction manual that accompanies the pad can also be provided that bears instructions regarding the method set forth herein. In any case, the temperature of the pad 12 can be controlled by a controller 18 in accordance with principles set forth in the external pad patents to exchange heat with the patient 14, including to establish normothermia in a febrile patient and to induce therapeutic mild or moderate hypothermia in the patient in response to the patient presenting with, e.g., cardiac arrest, myocardial infarction, stroke, high intracranial pressure, traumatic brain injury, or other malady the effects of which can be ameliorated by hypothermia.

When hypothermia is induced in the patient, the patient may shiver, and it may be desirable to counter the shivering. The patient may be covered by a blanket but more preferably is treated by injecting, into the patient's bloodstream through an intravenous line 20, one or more anti-shivering substances 22 that can be contained in a source 24 of anti-shivering drugs. The source 24 may be, without limitation, a syringe or IV bag or other source. In non-limiting embodiments the substance 22 may be any one of the substances disclosed in the anti-shivering patents.

FIG. 2 shows that at block 26, the patient presents with symptoms that are diagnosed as indicating hypothermia, e.g., the patient may be diagnosed with cardiac arrest, myocardial infarction, stroke, high intracranial pressure, traumatic brain injury, or other malady the effects of which can be ameliorated by hypothermia. At block 28, the pad 12 is placed on the external skin of the patient, and at block 30 hypothermia is induced in accordance with the external pad patents. However, in accordance with the present invention, instead of allowing the patient to shiver, at block 32 the anti-shivering substance 22 or other therapy (e.g., warming blanket) is administered to the patient to counter the shivering.

While the particular SYSTEM AND METHOD FOR REDUCING SHIVERING WHEN USING EXTERNAL COOLING PADS as herein shown and described in detail is fully capable of attaining the above-described objects of the invention, it is to be understood that it is the presently preferred embodiment of the present invention and is thus representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more”. It is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. Absent express definitions herein, claim terms are to be given all ordinary and accustomed meanings that are not irreconcilable with the present specification and file history.

What is claimed is:

1. A method for treating a patient, comprising:
disposing at least one heat exchange pad against the external skin of the patient;
exchanging heat with the patient using the pad to lower the body temperature of the patient; and
administering at least one anti-shivering agent to the patient.

2. The method of claim 1, wherein the administering act is undertaken by injecting at least one substance into the bloodstream of the patient.

3. The method of claim 1, further comprising diagnosing the patient as having had cardiac arrest, prior to the act of exchanging heat.

4. The method of claim 1, further comprising diagnosing the patient as having had a myocardial infarction, prior to the act of exchanging heat.

5. The method of claim 1, further comprising diagnosing the patient as having had a stroke, prior to the act of exchanging heat.

6. A system, comprising:
at least one pad configured for external placement on the skin of a patient and operable to cool the patient to induce hypothermia in the patient; and
at least one substrate associated with the pad and bearing instructions to administer at least one anti-shivering agent to the patient.

7. The system of claim 6, wherein the substrate is a label.

8. The system of claim 6, wherein the substrate is established by an instruction manual.

9. The system of claim 6, further comprising a temperature control system engaged with the pad to establish a temperature.

10. The system of claim 6, further comprising a source of the anti-shivering agent.

11. A method, comprising:
providing instructions to a medical practitioner, the instructions including:
placing at least one heat exchange pad against the skin of a patient;
inducing hypothermia in the patient using the pad; and
countering shivering in the patient.

12. The method of claim 11, wherein the instructions for countering shivering include administering at least one anti-shivering agent.

13. The method of claim 11, wherein the instructions further include diagnosing the patient as having had a cardiac arrest.

14. The method of claim 11, wherein the instructions further include diagnosing the patient as having had a myocardial infarction.

15. The method of claim 11, wherein the instructions further include diagnosing the patient as having had a stroke.
